(19) United States
(12) Patent Application Publication
(10) Pub. No.: US 2019/0049433 A1
(43) Pub. Date: Feb. 14, 2019

(54) ELECTRODE CONFIGURATION SCHEME FOR ELECTROPHYSIOLOGICAL TESTING DEVICES

(71) Applicant: MOBILEMEDTEK HOLDINGS, INC., Louisville, KY (US)

(72) Inventors: Bryan EHRET, Crestwood, KY (US); David KENNEDY, Georgetown, IN (US); Tyler Martin, Louisville, KY (US); Galen Powers, Louisville, KY (US); Steve Upchurch, Georgetown, IN (US); Olivia Ryan, Louisville, KY (US)

(21) Appl. No.: 16/059,458

(22) Filed: Aug. 9, 2018

Related U.S. Application Data
(60) Provisional application No. 62/542,999, filed on Aug. 9, 2017.

(51) Int. Cl. G01N 33/50; B01L 3/00; G01N 33/483

(52) U.S. Cl. CPC G01N 33/5014; B01L 3/5085; A61B 2018/1467; B01L 2300/0645; G01N 33/4836

(57) ABSTRACT

A device and system allows for simplified connection, disconnection, and mitigation of signal interference for electrophysiological testing. One or more electrophysiological electrodes are attached to the patient. These electrodes are connected to leads which are bundled based on region with a single connector associated with each bundle. The bundles are individually keyed and colored for ease of connection and disconnection. Leads within a bundle are organized through the use of a retainer. Electrode bundles are connected to a junction box within proximity to the patient. A single cable connects the junction box to an electrophysiological testing and monitoring interface. A system for visualizing lead bundle data, organizing individual electrode data by the lead bundles that they are associated, facilitates a workflow allowing for rapid and simple channel identification in association with specific electrode regions.

This application claims priority to U.S. Provisional Patent Application Ser. No. 62/542,999, filed Aug. 9, 2017, the disclosure of which is incorporated herein by reference.

### FIELD OF INVENTION

This invention is directed to electrophysiological testing, specifically, to electrode configuration and electrical connection of one or more electrophysiological testing wires between a subject and an electrophysiological device with user interface.

### BACKGROUND OF THE INVENTION

A multitude of electrophysiological tests are currently available for medical professionals to perform on subjects in order to analyze the electrical processes of the body, including electroencephalography (EEG), electromyography (EMG), nerve conduction studies (NCS), evoked potentials (EP), electrocardiography (ECG/EKG), and Holter monitoring. Electrophysiological tests detect electrical activity transduced to the skin's surface via an electrode frequently attached by a substance such as colloidal gel or paste. This information is currently transmitted through individual cables to a computer interface that amplifies the signal and may filter the signal which has been transmitted for analysis by a trained medical professional.

Specifically, EEG is a non-invasive test which is widely used to study electrical activity of the brain. EEG has been used to assess many different medical issues, including but not limited to epileptic seizures, locating areas of damage following head injury or stroke, and investigate sleep disorders. EEG utilizes a number of electrodes that measure electrical activity in multiple areas of the brain, including frontal, central, posterior, temporal and occipital regions. There are several electrode configurations currently used by medical professionals for EEG, including the 10/20 and 10/10 electrode systems. To determine the placement of electrodes, measurements must be taken between cranial structures including the nasion, preauricular points, and inion. From these points measurements of 10% and 20% must be determined for electrode placement. This typically produces between 21 and 329 points of connection which must be individually connected to a Brain Computer Interface (BCI) or electrophysiological testing device for amplification and analysis by medical professionals. During or just prior to EEG testing there are occasions that may require the subject to be disconnected from the BCI. By utilizing multiple cables individually attached to a BCI the mobility of a subject is limited and the ability of a medical professional to perform tests, such as MRI, is hindered at the expense of set up time.

EEG readings are highly sensitive and subject to artifacts due to movement and broken wire contacts. These artifacts are amplified upon transmission to an electrophysiological testing device. It becomes imperative to limit such incidents during subject testing to appropriately analyze data. Currently the process involves attaching individual cables to a BCI which can increase the potential for cable movement or broken wire contacts, which must be replaced. Furthermore, troubleshooting bad electrical connections is complex and hindered by the lack of intuitive links between the software user interface and the actual electrode connections on the patient's scalp.

Methods to combat the difficulties associated with EEG cable organization and simplify the connections have been pursued. Methods have been produced to simplify the disconnection process without simplifying the initial system connection scheme. Other methods have been produced which facilitate the ease and placement of electrodes on the head of a patient which may sacrifice specificity and test viability. Others methods of electrode placement facilitate the ease of connection to a BCI but maintain the number of lead contacts or lead connections requiring a technician for junction to head boxes. All of these techniques provide benefits which sacrifice either ease of test performance or accuracy of electrophysiological tests.

A system must be provided which will allow a medical professional to simply and freely attach and detach these cables from the patient for specific situations which require such an application. This system must be associated with a user interface scheme that is easily interpreted for troubleshooting electrode problems to facilitate better workflow of placing and verifying electrodes. A system must be provided which allows medical professionals to perform electrophysiological testing which limits the production of unintended abnormalities during testing without affecting the sensitivity of the device. This system should not affect the viability of test results.

### SUMMARY OF THE INVENTION

One object of the present disclosure is to provide a device and system for simplified connection, disconnection, and mitigation of signal interference for an electrophysiological testing device. One or more electrophysiological electrodes and leads may be attached to the patient. The electrode leads may be bundled based on anatomical regions and may terminate at a single connector, which may be individually keyed and/or colored for ease of connection and disconnection to a junction box. Electrode bundles may be connected to a single junction box within proximity to the patient. A single cable may connect the junction box to an electrophysiological testing and monitoring interface, such as a BCI. The associated user interface may maintain the correlation between color code, anatomical placement, and the acquired signal as part of a referential montage to facilitate rapid user interpretation. Together this device and system may generate a means to perform electrophysiological testing that easily connects and disconnects from a medical testing device and promotes faster detection and location of electrode signal abnormalities, all while maintaining the viability of the test results.

In one embodiment, an apparatus is provided for use in association with a plurality of electrodes and an electrophysiological testing device for supporting a plurality of electrode configurations on a patient. The apparatus may comprise a plurality of bundles, each of the bundles comprising a plurality of electrode leads, each of the electrode leads being adapted to transmit electrophysiological signals from one of the plurality of electrodes, wherein each of the plurality of bundles terminates at a single keyed connector. The apparatus may further comprise a junction box including a plurality of keyed receivers, each of the keyed receivers adapted to pair with and receive a corresponding keyed connector and a cable adapted to connect the junction box to the electrophysiological testing device. Each of the keyed receivers of the junction box may be adapted to receive the electrophysiological signals from the corresponding keyed connector, and the cable may be adapted to transmit the electrophysiological signals received at the junction box from each of the plurality of bundles to the electrophysiological testing device.

Each of the plurality of bundles may be identified by a different color. In another aspect, each of the plurality of bundles may be adapted for connection to a specific anatomical region on the patient. Each of the keyed receivers may be color coded to a respective one of the plurality of bundles, the color code comprising a different color corresponding to each pair of bundle and keyed receiver.

The plurality of electrode leads within at least one of the plurality of bundles may be of different lengths. The different lengths of the plurality of electrode leads within the at least one of the plurality of bundles may comprise at least one shorter length for a first electrode lead adapted for attachment to a back of a head of the patient and at least one longer length for a second electrode lead adapted for attachment to a front of the head of the patient. In another aspect, the different lengths of the plurality of electrode leads within the at least one of the plurality of bundles may comprise at least one shorter length for a first electrode lead adapted for attachment to a front of a head of the patient and at least one longer length for a second electrode lead adapted for attachment to a back of the head of the patient. In a further aspect, the plurality of electrodes within at least one of the plurality of bundles may all be the same length.

In another aspect, at least one retainer may be provided, said retainer connecting the plurality of electrode leads within at least one of the bundles at a longitudinal location between the electrodes and the keyed connector. The retainer may be adapted to slide longitudinally along the at least one bundle. In one aspect, a plurality of retainer may be provided, each of the plurality of retainers being attached to one of the plurality of bundles, wherein each of the plurality of bundles along with its corresponding retainer is identified by a different color.

In another embodiment, an apparatus is provided for use in association with a plurality of electrodes and an electrophysiological testing device for supporting a plurality of electrode configurations on a patient. The apparatus may comprise a plurality of electrode leads grouped into a plurality of bundles, wherein each of the plurality of bundles corresponds to a different anatomical region on the patient, wherein each of the plurality of bundles includes at least two of the electrode leads that terminate at a single connector, and wherein each of the electrode leads is adapted to transmit electrophysiological signals from one of the plurality of electrodes to a corresponding single connector. In addition, the apparatus may comprise a junction box including a plurality of receivers, each of the receivers adapted to be paired with the connector of one of the bundles and a cable adapted to connect the junction box to the electrophysiological testing device. Each of the receivers of the junction box may include an identifier that is coded to only one of the plurality of bundles. Each of the receivers of the junction boxy may be adapted to receive the electrophysiological signals from the corresponding paired connector. In another aspect, the cable may be adapted to transmit the electrophysiological signals received at the junction box from each of the plurality of bundles to the electrophysiological testing device.

The identifier may comprise a color, such that each of the receivers is color coded to only one of the bundles.

In another aspect, each connector of each bundle may be a different shape, and the identifier may comprise a shape, such that each of the receivers is shaped to correspond to the shape of only connector of only one of the bundles.

A plurality of retainers may be provided, each retainer adapted to receive and maintain relative position of the electrode leads within a given bundle. In a further aspect, the identifier may comprise color, such that each of the retainers includes a color that matches a color of its corresponding bundle, and each of the receivers may be color coded to only one of the bundles.

In a further embodiment, a method is disclosed for evaluating electrophysiological signals from a patient. The method may comprise attaching a plurality of electrodes to the patient, the plurality of electrodes being grouped according to a plurality of anatomical regions and adapted to transmit the electrophysiological signals from the patient. The method may further include providing a plurality of bundles of electrode leads, wherein each bundle comprises at least two electrode leads that terminate at a single connector, and wherein each bundle corresponds to a single one of the anatomical regions. In addition, the method may include connecting each of the electrode leads of a first bundle to electrodes of a first of the anatomical regions, and each of the electrode leads of a second bundle to electrodes of a second of the anatomical regions. A junction box may be provided, the junction box including a plurality of receivers color coded to correspond to a single one of the plurality of bundles and further including a cable adapted for pairing with an electrophysiological testing device. The method may include connecting the single connector of each of the plurality of bundles to a corresponding color coded receiver, pairing the cable of the junction box with the electrophysiological testing device, and displaying on the electrophysiological testing device the electrophysiological signals of the patient.

In one aspect, the electrophysiological signals may be displayed in groups according to anatomical regions. In another aspect, the electrophysiological signals may be displayed in groups according to bundle. In a further aspect, the electrophysiological signals may be displayed in groups according to the color coding of the bundles and receivers.

### BRIEF DESCRIPTION OF DRAWINGS

FIG. 1 is a schematic of a system for connecting electrodes to a patient for collection and display of electrophysiological data;

FIG. 2 illustrates one embodiment of a bundle of leads and electrodes of the system of FIG. 1;

FIG. 3 illustrates a further embodiment of a bundle of leads and electrodes of the system of FIG. 1;

FIG. 4 is a schematic of a plurality of bundles of leads and electrodes connected to a patient in a 10/20 configuration for EEG;

FIG. 5A is a schematic of placement of a plurality of groupings of electrodes connected to a patient in a 10/10 configuration for EEG;

FIG. 5B is a schematic of a plurality of bundles of leads adapted for connecting to the plurality of groupings of electrodes of FIG. 5A;

FIG. 6 is a junction box for connection of lead bundle leads corresponding to 10/20 electrode placement;

FIG. 7 is a junction box for connection of lead bundle leads corresponding to 10/10 electrode placement;

FIG. 8 illustrates a user interface organizing lead bundle data utilizing specific identifiers associated with electrode placement region;

FIGS. 9A and 9B illustrate one embodiment of a retainer; and

FIGS. 10A and 10B illustrate a second embodiment of a retainer.

### DETAILED DESCRIPTION OF THE INVENTION

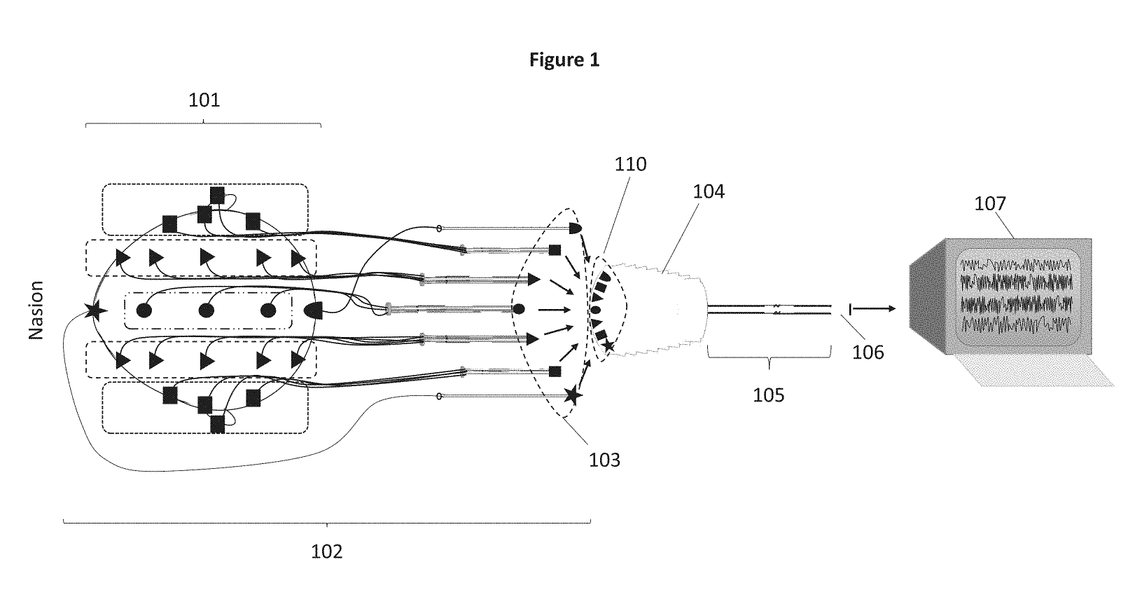

FIGS. 1-8 illustrate various embodiments of a device and system for simplifying connection and detachment of a patient to an electrophysiological testing device while maintaining test viability while connected. The embodiments provide a system which receives electrophysical signal through individual electrodes 101 attached to leads 102 which may be grouped by anatomical region and terminate at individually keyed connectors 103.

These connectors may interact with individually keyed receivers 110 of a junction box 104, with each keyed connector 103 corresponding to a particular keyed receiver 110. For example, each keyed connector 103 may correspond in shape to an individual keyed receiver 110, but not to other keyed receivers. The junction box 104 may relay individual signals through a single cable 105 and connector 106 to an electrophysiological device with user interface 107. This user interface 107 may organize and display electrophysiological data based on lead groupings for ease in identifying specific electrophysiological signal. One skilled in the art will appreciate the disclosed material is illustrative and should not be construed as limited to an individual electrophysiological test or electrode configuration. Modifications to embodiments described in this document, and other embodiments, will be evident to those of ordinary skill in the art after review of information provided. The described embodiments provided in this document are not to be considered limiting.

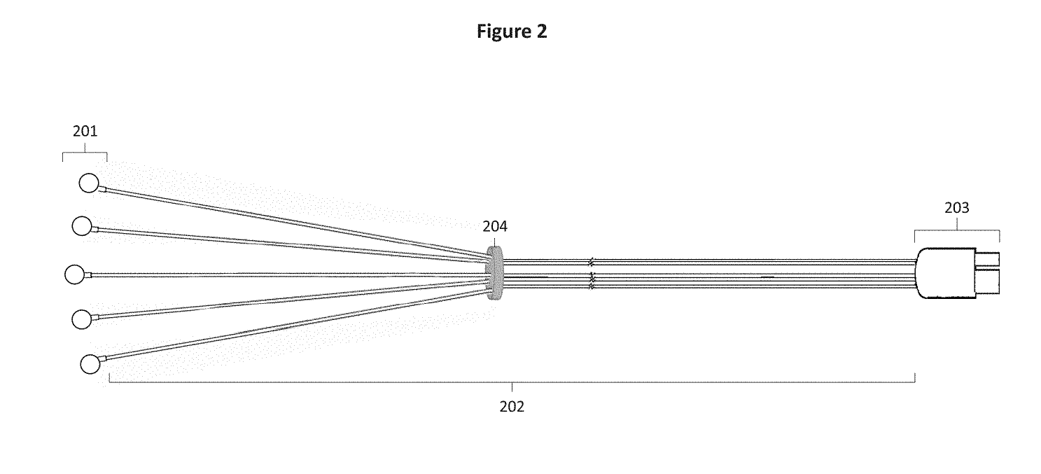

FIG. 2 is a diagram illustrative of an embodiment of one or more electrophysiological monitoring electrodes 201, which may be grouped by one or more leads 202 of equal length terminating at a single specifically keyed connector 203. One or more retainers 204 may be provided for connecting and retaining the individual leads 202 together in a single bundle.

The one or more electrophysiological electrodes 201 may attach to patients using an adhesive or other form of releasable connection. These electrodes 201 may be attached to or detachable from the individual leads 202. In some embodiments, the electrodes 201 may be permanently affixed to respective lead wires 202. One or more electrodes 201 may be attached to patient through mechanisms comprising but not limited to colloidal gel or paste and/or reusable or electrode heads such as disposable electrode cups or ear clips.

Leads 202 may comprise an individual conductive wire or cable. The location of these electrodes 201 and leads 202 attached to a specifically keyed connector 203 is dependent on the type of electrophysiological monitoring desired.

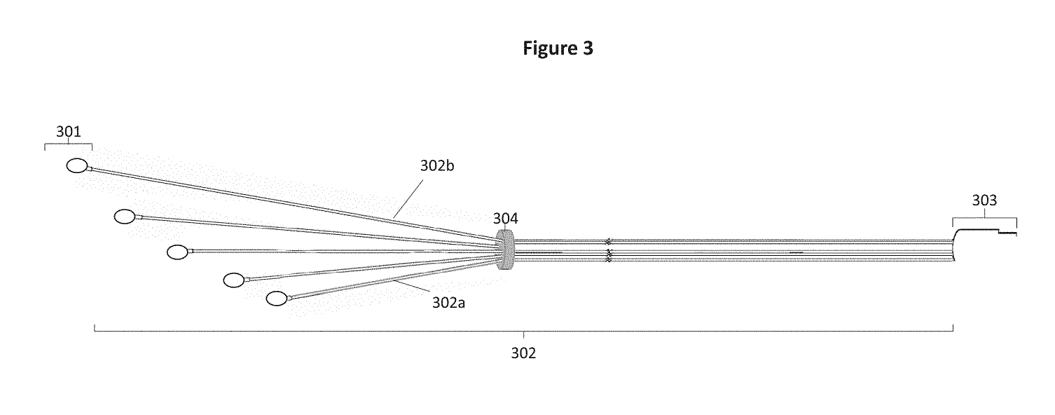

FIG. 3 illustrates a plurality of electrophysiological electrodes 301 which may be connected to one or more of a plurality of leads 302 of variable length. The plurality of leads 302 may terminate at a single specifically keyed connector 303. As with the embodiment of FIG. 2, a retainer 304 may be provided for connecting and retaining the individual leads 302 together in a single bundle.

A length of each of the individual leads 302 may correspond to a respective location or placement of one or more electrodes 301 upon the patient in a given test. For example, a shorter lead 302a may be adapted for electrodes placed toward the inion and a longer lead 302b may be adapted for electrodes placed towards the nasion. Alternatively, lead length may correspond to probe placement with shorter lead lengths associated with electrodes placed toward the nasion and longer lead lengths correspond to electrodes placed near the inion. The number of electrodes 301 and leads 302 attached to each specifically keyed connector 303 is dependent on the type of electrophysiological electrode configuration to be used.

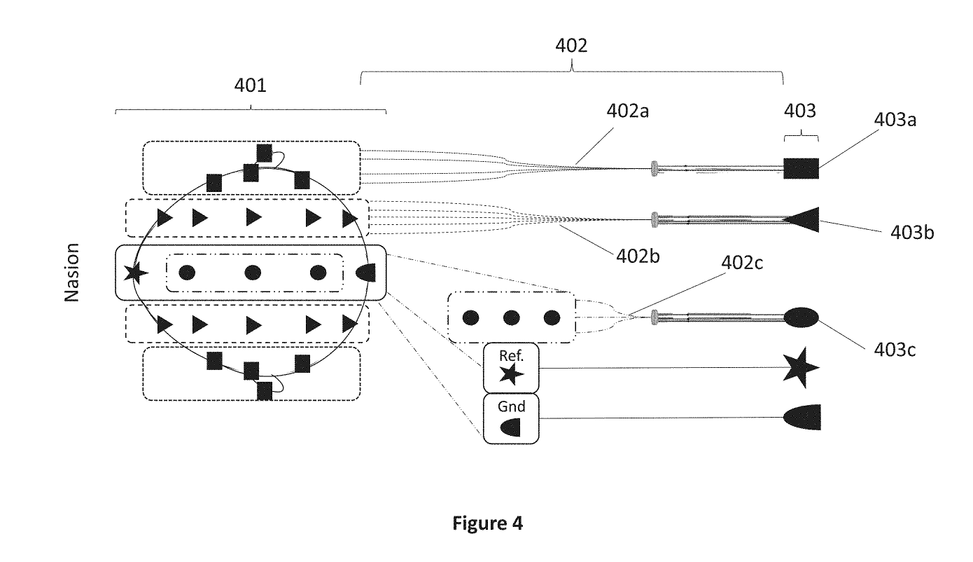

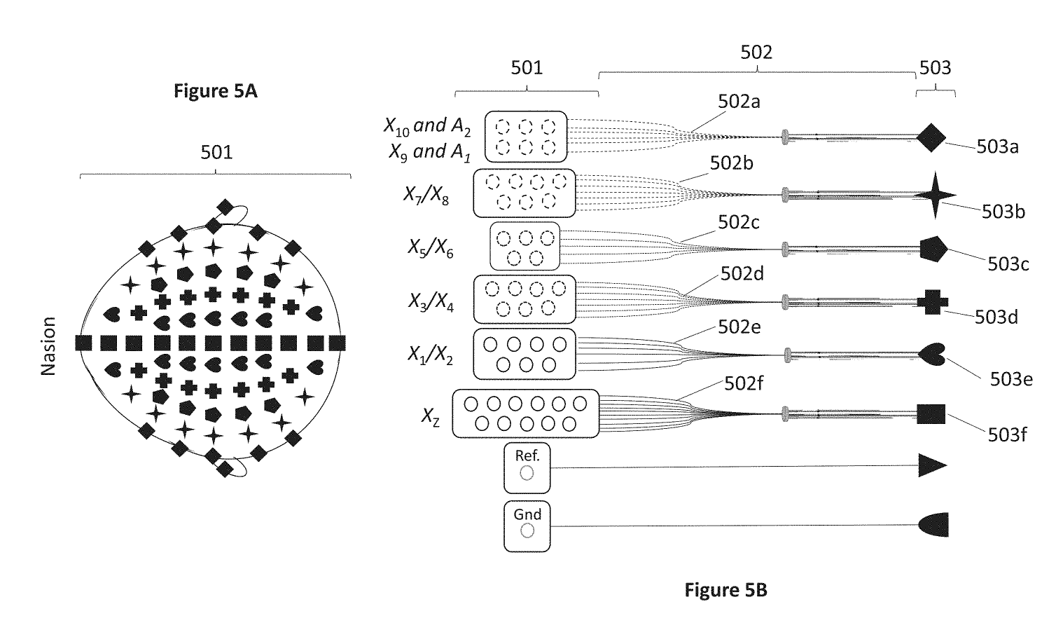

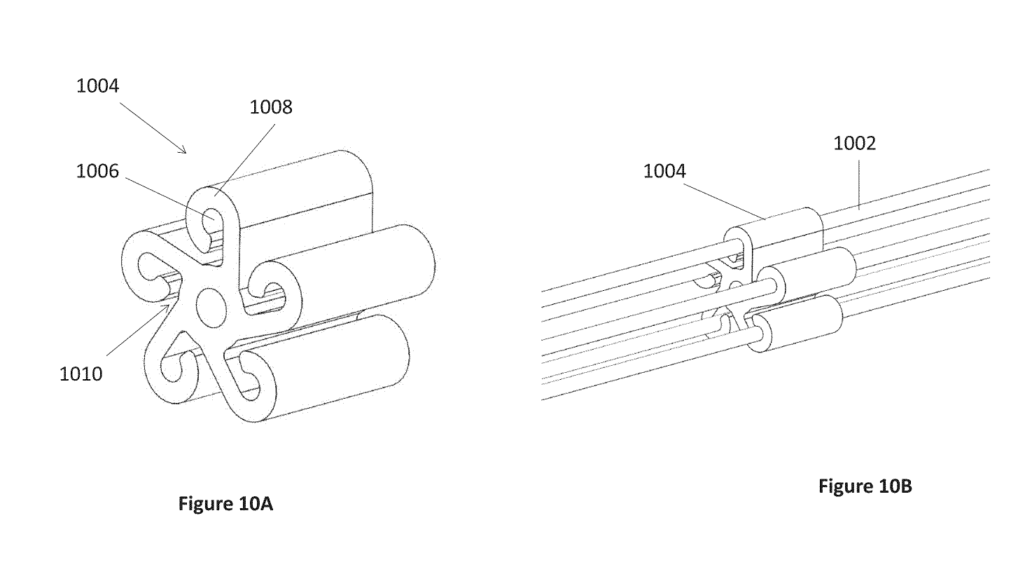

With reference to FIGS. 4 and 5B, a plurality of leads 402, 502 may terminate at a plurality of different specifically keyed connectors 403, 504. Electrophysiological electrode leads 402, 502 may be grouped or bundled by region of connection, such as brain region or alphanumeric region, corresponding to multiple electrode configurations, such as different EEG configurations comprising but not limited to 10/20, as illustrated in FIGS. 4, and 10/10, as illustrated in FIGS. 5A-5B. Electrode leads 402, 502 may comprise wires or cables.

In one aspect, electrode leads 402 may be grouped into specific bundles, such as bundles 402a, 402b, 402c of FIG. 4 or bundles 502a, 502b, 502c, 502d, 502e, 502f of FIG. 5B. These bundles may be specifically colored corresponding to specific electrode regions including but not limited to brain midline, parasagittal, temporal regions, and alphanumeric regions. Various embodiments of the system may allow certain keyed connectors 403, 503 to be similarly colored and keyed such that they are interchangeable with respect to anatomical symmetries.

For example, a first bundle 402a of a 10/20 EEG configuration may be a first color, such as blue, and its corresponding first keyed connector 403a may also be blue. Blue may represent a first anatomical placement corresponding to a first brain region. A second bundle 402b may be a second color, such as red, and its corresponding second keyed connector 403b, which may be different in shape from the first keyed connector 403a, may also be red. Red may represent a second anatomical placement corresponding to a second brain region. Similarly, a third bundle 402c may be a third color, such as yellow, and its corresponding third keyed connector 403c, which may be different in shape from both the first and second keyed connectors 403a, 403b, may also be yellow. Yellow may represent a third anatomical placement corresponding to a third brain region.

In a further aspect, the length of leads 402, 502 of a given bundle may correspond to an anatomical placement of the electrodes 401,501 associated with the leads of said bundle. For example, a first bundle adapted for and keyed for placement on a left temporal region may be longer than a second bundle adapted for and keyed for placement on a brain midline region, or vice versa.

In another aspect, the length of leads 402, 502 within a given bundle may vary such that the lengths of the leads correspond to an anatomical placement of the electrodes 401, 501 associated within said leads within said bundle. For example, a first lead within a first bundle 502a which corresponds to an electrode adapted for placement in a left temporal region nearer the nasion may be longer than a second lead within the first bundle which corresponds to an electrode adapted for placement in the left temporal region nearer the inion.

With further reference to FIG. 4, an embodiment is illustrated in which a plurality of electrodes 401 and their corresponding leads 402 are arranged for utilization in a 10/20 electrode configuration for EEG. As illustrated, the leads 402 are grouped in bundles 402a, 402b, 402c, with each bundle terminating in an individually keyed connector 403a, 403b, 403c. Each bundle identifies and represents an anatomical placement corresponding to brain region, such as frontal, temporal, parietal, and occipital regions. FIG. 4 depicts an example grouping of adjacent electrodes 401 which include between one and five electrodes and leads 402 per individual grouping providing seven individually grouped regions. Five of these regions correspond roughly to a central line from nasion to inion connected to bundle 402c, left and right parasagittal regions, the right parasagittal region connected to bundle 402b, and left and right temporal regions, the right temporal region connected to bundle 402a. The electrodes 401 for the left parasagittal region and the left temporal region are illustrated as well, and while corresponding leads are not illustrated for these regions, it is understood that each of these sets of electrodes connects to its own keyed bundle of leads in the same way as is illustrated with respect to the right parasagittal region and the right temporal region. In addition to these five individually grouped regions, a reference and ground electrode are both provided, which are illustrated as being placed along the center line, each of which being connected to its own lead or bundle of leads. Each of the individual bundles includes its own keyed connector 403, for a total of seven keyed connectors.

With reference to FIG. 5A, a plurality of electrodes 501 are illustrated as connected to a patient according to a 10/10 electrode configuration for EEG. The electrodes 501 of FIG. 5A are grouped according to anatomical placement. As illustrated, the groupings include between one and eleven electrodes. Each group as shown corresponds to illustrated shapes, including diamonds, stars, pentagons, crosses, hearts, and squares, such that each shape corresponds to the electrodes of a given anatomical region. In one aspect, each shape may correspond to or is representative of an individual color, such that a single color corresponds to a given anatomical region.

The anatomical regions may correspond to alphanumeric coordinates, such as nomenclature understood to correspond to standard 10/10 electrode configurations. For instance, the regions may correspond to regions including but not limited to frontotemporal, frontocentral, temporal-posterior temporal, centroparietal, posterior temporo-occipital, parieto-occipital, anterior frontal, occipital, and temporal, and numerical designations correspond to standardized 10/10 nomenclature. As illustrated in FIGS. 5A and 5B, there are thirteen individually grouped regions. As will be understood with reference to FIG. 5B, the nomenclature may include the letter “X” or the letter “A” and a given number, which may correspond to a particular anatomical location.

With further reference to FIG. 5B, a plurality of leads 502 are grouped according to bundles 502a, 502b, 502c, 502d, 502e, 502f. Each individual bundle corresponds to a particular set of electrodes 501, which may be one of the groups of electrodes as illustrated in FIG. 5A. Each of the bundles may terminate in a particular keyed connector 503a, 503b, 503c, 503d, 503e, 503f. For example, as illustrated, bundle 502a includes a plurality of leads connected to a single keyed connector 503a, which is illustrated as a diamond. The leads of bundle 502a may be connected to the electrodes of FIG. 5A of corresponding shape, namely the electrodes 501 shaped like diamonds. Accordingly, the bundle 502a may be specifically adapted for connection to the patient via the respective electrodes at the anatomical region of the patient which corresponds to those electrodes.

One or more of the leads of bundle 502a, the keyed connector 503a, and the corresponding electrodes 501 of FIG. 5A may comprise a first color, which may correspond to or identify a first anatomical region as described above. For example, the color blue may correspond to a first anatomical region, such as the right temporal region, and therefore one or more of the leads of the first bundle 502a, the first keyed connector 503a, and the electrodes 501 of FIG. 5A which are illustrated as diamond-shaped may comprise the color blue. Similarly, a second color, such as red, may correspond to or identify a second anatomical region, such as the midline region, and therefore one or more leads of a second bundle 502f, the second keyed connector 503f, and the electrodes 501 of FIG. 5A which are illustrated as rectangles may comprise the color red.

In a further aspect, a retainer 204, 304 may be provided for organizing, separating, managing and retaining electrode lead bundles, such as is illustrated in FIGS. 2 and 3. The retainer may comprise a flexible ring, notched clip, a band cinch, a sliding band, such as may be made of nylon or Teflon tubing, or a wire cinch. This retainer 204, 304 may be adapted to be move along a longitudinal axis of the leads of individual bundles 202, 302 for adjustment through sliding, rolling or a combination of the two. The retainer may be adapted for maintaining the leads of a given bundle within a fixed relative radial position within the retainer, such that the leads will not get tangled with one another, even upon movement of the retainer along the longitudinal axis of the leads of the bundle. In one aspect, the retainer may be adapted to allow the wires to be securely snapped into the retainer.

Movement of the retainer along the longitudinal axis of the bundle may allow the retainer to help reform or modify a condition of the bundle. For example, the retainer 204, 304 may be mobile along the leads of the bundles 202, 302, thereby limiting loose cable length. In another aspect, the retainer may be colored corresponding to color of its corresponding bundle 202, 302.

In one embodiment, the retainer 204, 304 may be a notched clip. The notched clip may include notches or channels adapted to retain one or more leads of a given bundle to keep them arrayed in a specific form such that the wires are not twisted or tangled. The retainer 204, 304 may be configurable for any number of leads within or between bundles 202, 302.

In one aspect, a plurality of retainers 204, 304 may be placed along the length of a given bundle 202, 302. The retainers 204, 304 may be permanent or removable. It should be appreciated by one skilled in the art that the retainer 204, 304 may be made of any number of flexible or rigid materials.

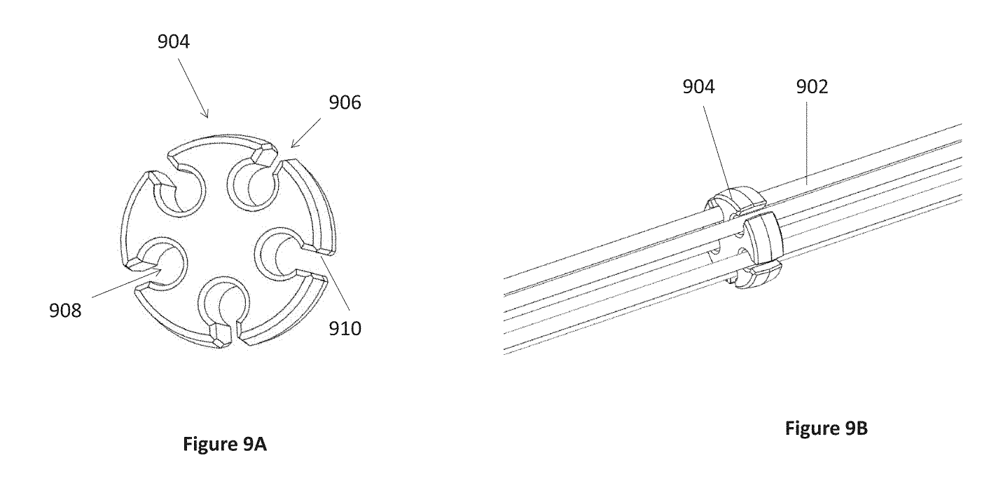

With further reference to FIGS. 9A-9B, one embodiment of a retainer 904 is illustrated. The retainer 904 may include one or more receivers 906 adapted to receive the electrode leads 902 of a given bundle of leads. As illustrated, a plurality of receivers 906 may be spaced around the retainer 904, such that a plurality of electrode leads 902 may be retained in relative circumferential position with respect to each other around the retainer 904.

In one aspect, the receivers 906 may be adapted to form an interference fit with the electrode leads 902. Accordingly, the receivers 906 may comprise an enlarged recess 908 in which the electrode leads 902 may be placed. In another aspect, the receivers 906 may comprise a mouth 910 which is smaller than the enlarged recess 908. The mouth 910 and enlarged recess 908 may be sized such that a diameter of the electrode lead 902 is larger in diameter than, or no smaller than, the mouth 910, such that the electrode leads 902 may be snapped into and the enlarged recess 908 through the mouth 910, and retained therein by the mouth, which is no greater than the diameter of the electrode lead 902. The enlarged recess 908 may be at least as large in diameter as the diameter of the electrode lead 902, such that the retainer 904 may be allowed longitudinal movement along the electrode leads 902 when said leads are retained within the recesses 908. In one aspect, the diameters of the recesses 908 may be approximately equal to the diameters of the electrode leads 902. Accordingly, friction may hold the retainer 904 at a given longitudinal position along the electrode leads 902. Said friction may be overcome by applying a force to the retainer 904 in a direction along the longitudinal axis of the electrode leads, so that the retainer 904 may be moved along said electrode leads.

As shown in FIGS. 10A-10B, a second embodiment of a retainer 1004 is illustrated. The retainer 1004 may include a plurality of arms 1008, which may include receivers 1006 adapted to receive the plurality of electrode leads 1002 of a given electrode lead bundle. The receivers 1006 may be adapted to form an interference fit with the electrode leads 1002.

As illustrated, the plurality of arms 1008 may extend radially outward from a central body. In one aspect, the arms may form a floriform configuration. The receivers 1006 may include a narrowed mouth 1010, which may allow for the interference fit with the electrode leads. In a further aspect, the arms 1008 may be adapted to wrap around a given electrode lead 1002, with the narrowed mouth 1010 adapted to hold the electrode lead within the receiver 1006. Such a configuration may require a force to be applied to open the receiver 1006 of each arm to insert or remove an electrode lead from the receiver. The retainer 1004 may be adapted to recoil and wrap around the electrode lead upon removal of said force. For example, the retainer 1004 may comprise a resilient material that may temporarily be deformed by a force for insertion or removal of the electrode lead 1002, and then retake its original shape upon removal of the force. Similarly to the embodiment of FIGS. 9A-9B, the receivers 1006 may be sized in diameter similarly to the diameter of the electrode leads, such that friction may hold the retainer 1004 in place, but longitudinal force applied to the retainer 1004 along the electrode leads may allow for movement of the retainer along the longitudinal axis of the electrode leads.

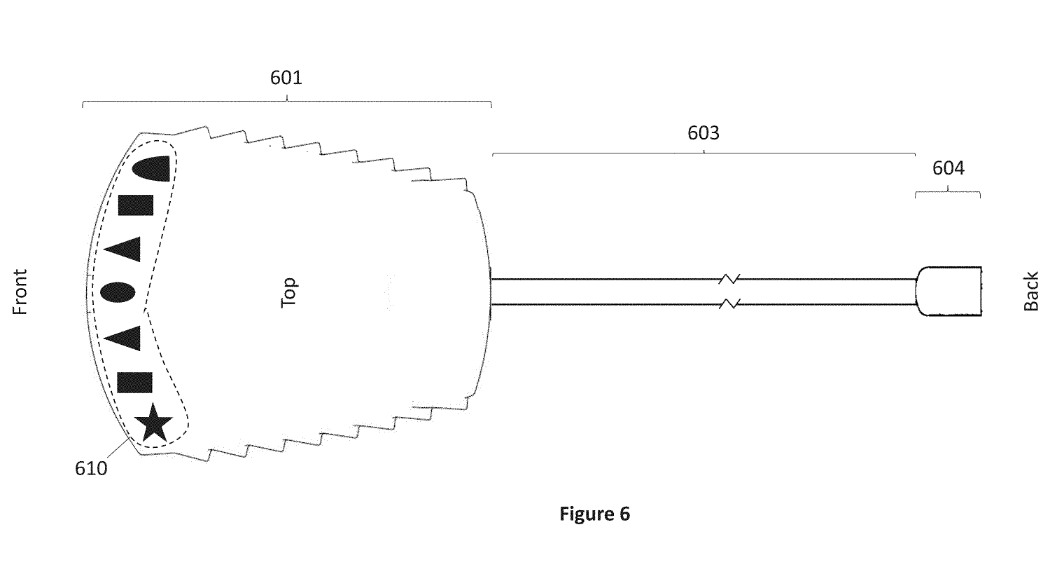

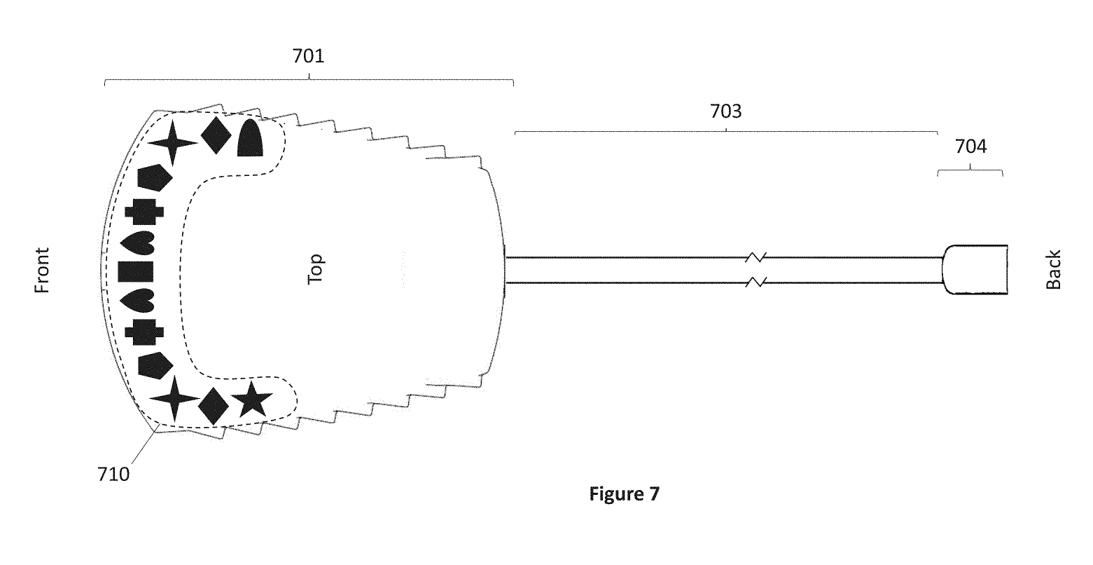

With reference to FIGS. 6 and 7, a junction box 601, 701 may be provided, which may include one or more specifically keyed receivers 610, 710. These keyed receivers may correspond to specific keyed connectors 403, 503. The junction box 601, 701 may utilize a single cable 603, 703 and a connector 604, 704 for interaction with an electrophysiological testing device. In other aspects, a plurality of cables and/or connectors may connect the junction box to the electrophysiological testing device.

The keyed receivers 610, 710 may correspond to specific electrode lead bundles 402, 502. For example, the keyed receivers 610, 710 may be colored so as to correspond to a specific color of a given bundle 402, 502, as outlined above. The keyed receivers 610, 710 may be placed on the front or sides or on top of a housing of the junction box 601, 701. Keyed receivers 610, 710 may be located on the junction box such that their location may correspond to the anatomical location of electrodes of the corresponding bundles 402, 502.

In a further aspect, keyed receivers 610, 710 for left and right corresponding regions, such as parasagittal regions, may have the same identifiers or keying. For example, the electrodes 401, 501, the lead bundles 402, 502, the keyed connectors 403, 503, the corresponding keyed receivers (or any combination thereof) which correspond to the left temporal region may have the same identifier (e.g. color) and keyed configuration as those of the right temporal region. In another example, the identifier, such as color, of the electrodes, bundles, keyed connectors, keyed receivers (or any combination thereof) of the left and right similar anatomical regions may be the same, but the keyed configurations of left and right elements may be different. Alternately, both the identifier, such as color, and the keyed configurations of the electrodes, bundles, keyed connectors, keyed receivers (or any combination thereof) of the left and right similar anatomical regions may both be different from one another.

In use, the junction box 601, 701 may be placed within close proximity to the patient to eliminate length of electrode lead bundle length. One skilled in the art will appreciate that the junction box 601, 701 may be constructed of any one of a variety of materials such as molded plastic and/or metal, and may be configured according to various shapes or geometries.

As illustrated, FIG. 6 depicts an embodiment of a junction box 601 in which a signal originates at electrodes 401, is relayed through bundles 402 and through specifically keyed connectors 403 via specifically keyed receivers 610 being utilized for a 10/20 electrode configuration for EEG as described above. Specifically, keyed receivers 610 may correspond to the specifically keyed connectors 403 of the respective bundles 402 for a 10/20 electrode configuration. Keyed receivers 610 of junction box 601 may also be specifically labeled using an identifier, such as color coding, for coordination with a 10/20 configuration.

Similarly, FIG. 7 depicts an embodiment of a junction box 701 in which a signal originating at electrodes 501, is relayed through bundles 502 and through specifically keyed connectors 503 to specifically keyed receivers 710 being utilized for a 10/10 electrode configuration for EEG. Specifically keyed receivers 710 may correspond to the specifically keyed connectors 503 of bundles 502 for the 10/10 electrode configuration. Keyed receivers 710 of junction box 701 may also be specifically labeled using an identifier, such as color coding, for coordination with a 10/10 configuration.

The cable 603, 703 associated with the junction box 601, 701 may include one or more individual conductive signal wires or cables originating at the junction box 601, 701 and terminating at the connector 704 specific for relaying electrode signal to an electrophysiological monitoring interface including but not limited to a BCI, a computer terminal, and a display screen. The cable 603, 703 may be shielded with strip or braided conductive material and may be surrounded in protective material.

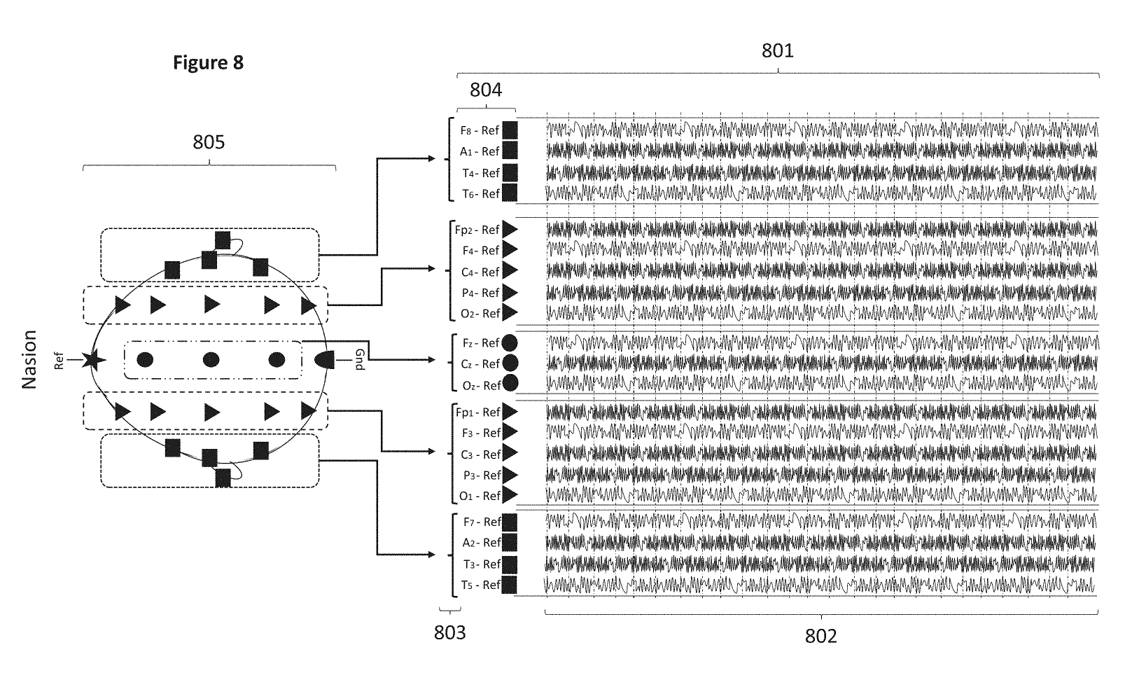

FIG. 8 depicts an electrophysiological testing system which is utilized to present data in a visual form via a medical computing interface 801, such as a BCI. Data representing electrophysiological signals 802 received from electrodes attached to patients may be organized and grouped based on the specific electrode and lead bundles, represented in FIG. 8 at 803. Graphic depiction of text and data may be depicted using an identifier 804, such as color coding, corresponding to specific lead bundle signal or anatomical region 805 for electrode attachment.

For example, a strip chart graph may be displayed in which data from bundles 803 are depicted and organized together and may be given specific identifiers 804 which contrast for ease of visualization. Color coding or other identifiers 804, such as shape, may coordinate with a coding which is also applied to electrodes 401, 501, bundles 402, 502, keyed connectors 403, 503, and/or keyed receivers 610, 710. Graphic depiction of text for data associated with specific lead bundles, such as identifiers 804, may also be associated with colored shapes for ease of visualization. Color coding or shape display may also change to indicate detection of specific signal patterns occurring in a data channel, such as change in impedance or other signal abnormality. The data channels corresponding to specific bundles 803 may be grouped with the electrode nearest the nasion represented as the first data channel while the electrode nearest the inion may be visualized as the last data channel. Accordingly, a workflow allowing for rapid and simple channel identification corresponding to specific electrode anatomical regions 805 may be facilitated.

With respect to the disclosed subject matter, the preferred patient is a warm-blooded mammal, with the most preferable being a human. As used herein the term “patient” is reflective of such preference.

With further respect to the disclosed subject matter, electrophysiological tests include any test which measures electrical potential of a patient utilizing electrodes specific for such assessment. Examples of electrophysiological tests include electroencephalography (EEG), electromyography (EMG), nerve conduction studies (NCS), evoked potentials (EP), electrocardiography (ECG/EKG), and Holter monitoring. The most preferred electrophysiological test is EEG. In other embodiments, however, one or more of these tests may be used in parallel and transmitted along with patient EEG data.

It will be understood that various details of the presently disclosed subject matter can be changed without departing from the scope of the subject matter disclosed herein. Furthermore, the foregoing description is for the purpose of illustration only, and not for the purpose of limitation. As such, total embodiments which may be utilized should not be limited by expressed embodiments of 10/10 and 10/20 electrode configurations.

1. An apparatus for use in association with a plurality of electrodes and an electrophysiological testing device for supporting a plurality of electrode configurations on a patient, the apparatus comprising:
a plurality of bundles, each of the bundles comprising a plurality of electrode leads, each of the electrode leads being adapted to transmit electrophysiological signals from one of the plurality of electrodes, wherein each of the plurality of bundles terminates at a single keyed connector; and
a junction box including a plurality of keyed receivers, each of the keyed receivers adapted to pair with and receive a corresponding keyed connector; and
a cable adapted to connect the junction box to the electrophysiological testing device;
wherein each of the keyed receivers of the junction box is adapted to receive the electrophysiological signals from the corresponding keyed connector, and wherein the cable is adapted to transmit the electrophysiological signals received at the junction box from each of the plurality of bundles to the electrophysiological testing device.

2. The apparatus of claim 1, wherein each of the plurality of bundles is identified by a different color.

3. The apparatus of claim 1, wherein each of the plurality of bundles is adapted for connection to a specific anatomical region on the patient.

4. The apparatus of claim 1, wherein the plurality of electrode leads within at least one of the plurality of bundles comprise different lengths.

5. The apparatus of claim 4, wherein the different lengths of the plurality of electrode leads within the at least one of the plurality of bundles comprises at least one shorter length for a first electrode lead adapted for attachment to a back of a head of the patient and at least one longer length for a second electrode lead adapted for attachment to a front of the head of the patient.

6. The apparatus of claim 4, wherein the different lengths of the plurality of electrode leads within the at least one of the plurality of bundles comprises at least one shorter length for a first electrode lead adapted for attachment to a front of a head of the patient and at least one longer length for a second electrode lead adapted for attachment to a back of the head of the patient.

7. The apparatus of claim 1, wherein the plurality of electrodes within at least one of the plurality of bundles are all the same length.

8. The apparatus of claim 1, further including at least one retainer connecting the plurality of electrode leads within at least one of the bundles at a longitudinal location between the electrodes and the keyed connector.

9. The apparatus of claim 8, wherein the retainer is adapted to slide longitudinally along the at least one bundle.

10. The apparatus of claim 8, wherein the at least one retainer comprises a plurality of retainers, each of the plurality of retainers attached to one of the plurality of bundles, wherein each of the plurality of bundles along with its corresponding retainer is identified by a different color.

11. The apparatus of claim 2, wherein each of the keyed receivers is color coded to a respective one of the plurality of bundles, said color code comprising a different color corresponding to each pair of bundle and keyed receiver.

12. An apparatus for use in association with a plurality of electrodes and an electrophysiological testing device for supporting a plurality of electrode configurations on a patient, the apparatus comprising:
a plurality of electrode leads grouped into a plurality of bundles,
wherein each of the plurality of bundles corresponds to a different anatomical region on the patient,
wherein each of the plurality of bundles includes at least two of the electrode leads that terminate at a single connector, and
wherein each of the electrode leads is adapted to transmit electrophysiological signals from one of the plurality of electrodes to a corresponding single connector;
a junction box including a plurality of receivers, each of the receivers adapted to be paired with the connector of one of the bundles; and
a cable adapted to connect the junction box to the electrophysiological testing device;
wherein each of the receivers of the junction box includes an identifier that is coded to only one of the plurality of bundles;
wherein each of the receivers of the junction box is adapted to receive the electrophysiological signals from the corresponding paired connector; and
wherein the cable is adapted to transmit the electrophysiological signals received at the junction box from each of the plurality of bundles to the electrophysiological testing device.

13. The apparatus of claim 12, wherein the identifier comprises a color, such that each of the receivers is color coded to only one of the bundles.

14. The apparatus of claim 12, wherein each connector of each bundle is a different shape, and wherein the identifier comprises a shape, such that each of the receivers is shaped to correspond to the shape of only connector of only one of the bundles.

15. The apparatus of claim 12, further including a plurality of retainers, each retainer adapted to receive and maintain relative position of the electrode leads within a given bundle.

16. The apparatus of claim 15, wherein the identifier comprises color, such that each of the retainers includes a color that matches a color of its corresponding bundle, and wherein each of the receivers is color coded to only one of the bundles.

17. A method of evaluating electrophysiological signals from a patient comprising:
attaching a plurality of electrodes to the patient, the plurality of electrodes being grouped according to a plurality of anatomical regions and adapted to transmit the electrophysiological signals from the patient;
providing a plurality of bundles of electrode leads, wherein each bundle comprises at least two electrode leads that terminate at a single connector, and wherein each bundle corresponds to a single one of the anatomical regions;
connecting each of the electrode leads of a first bundle to electrodes of a first of the anatomical regions, and each of the electrode leads of a second bundle to electrodes of a second of the anatomical regions;
providing a junction box including a plurality of receivers color coded to correspond to a single one of the plurality of bundles and further including a cable adapted for pairing with an electrophysiological testing device;
connecting the single connector of each of the plurality of bundles to a corresponding color coded receiver;
pairing the cable of the junction box with the electrophysiological testing device; and
displaying on the electrophysiological testing device the electrophysiological signals of the patient.

18. The method of claim 17, wherein the electrophysiological signals are displayed in groups according to anatomical regions.

19. The method of claim 17, wherein the electrophysiological signals are displayed in groups according to bundle.

20. The method of claim 167 wherein the electrophysiological signals are displayed in groups according to the color coding of the bundles and receivers.
